(19) United States
(12) Patent Application Publication
(10) Pub. No.: US 2005/0143283 A1
(43) Pub. Date: Jun. 30, 2005

(54) Activated protein c formulations

(75) Inventors: Ganapathy Gopalrathnam, Fishers, IN (US); Lihua Huang, Carmel, IN (US); Ralph Meredith riggin, Carmel, IN (US); Theodore Arsay Sheliga, Indianapolis, IN (US)

Correspondence Address: ELI LILLY AND COMPANY;PATENT DIVISION, P.O. BOX 6288, INDIANAPOLIS, IN, 46206-6288, US

(73) Assignee: Eli Lilly and Company, Indianapolis, IN (US)

(21) Appl. No.: 10/506,301

(22) PCT Filed: Feb. 27, 2003
(86) PCT No.: PCT/US03/05046

Related U.S. Application Data
(60) Provisional application No. 60/363,364, filed on Mar. 8, 2002.

(51) Int. Cl. A61K 38/17; A61K 31/7024; A61K 31/198

(52) U.S. Cl. 514/2; 514/53; 514/566

(57) ABSTRACT

The invention relates to pharmaccutical compositions of activated protein C with a chelating agent. Preferably, the formulation contains activated protein C, a chelating agent, a bulking agent, a buffer, and/or a salt with a reconstituted pH between about 5.5 5 and about 6.5. Alternatively, the chelating agent is added to the diluent used with the activated protein C pharmaceutical composition. The aPC pharmaceutical compositions, formulations, and uses of the invention have improved in-use stability.

### FIELD OF THE INVENTION

This invention is in the field of human medicine, particularly in the treatment of vascular disorders with activated protein C. More specifically, the invention relates to pharmaceutical compositions, formulations, and uses of recombinant human activated protein C.

### BACKGROUND OF THE INVENTION

Protein C is a serine protease and naturally occurring anticoagulant that plays a role in the regulation of homeostasis by inactivating Factors Va and VIIIa in the coagulation cascade. Protein C activation results from removal of a dodecapeptide at the N-terminus of the heavy chain, producing activated protein C (aPC) possessing enzymatic activity.

In addition to the enzymatic activities of aPC within the blood coagulation cascade, aPC also can degrade. Activated protein C degradation can lead to variants with anticoagulant activity similar to the full-length chain or to less active variants. According to Foster et al. (WO 91/09951), aPC can include active variants consisting of deletions from the full-length light chain. Foster indicated that such variants include from 149 to 152 amino acid residues and that these light chain deletions do not substantially alter the activity.

In contrast to Foster et al., Applicants discovered that the 1-149 light chain variant was less active while the 1-150, 1-151, and 1-152 variants were active. This less active variant leads to decreased functionality as an anticoagulant via diminished potency.

In order to administer aPC to patients, freeze dried formulations of aPC formulation are reconstituted with diluent, added to an intravenous infusion solution, and administered via intravenous transfusion over the course of many hours. This aPC in-use solution has shown decreased potency levels due to the presence of the less active 1-149 aPC C-terminal light chain variant. Therefore, minimizing the level of the 1-149 aPC C-terminal light chain variant degradation product is important in achieving a potent in-use aPC formulation.

Applicants discovered that the addition of a chelating agent to the diluent used with the aPC formulation or to the aPC formulation itself improves the solution stability of aPC. Linnau et al. (AU 9892449) teaches pharmaceutical preparations of at least two blood coagulation factors, including protein C, and involves the use of a chelating agent. Premature thrombin formation in the preparation is prevented if calcium ions are displaced by the presence of magnesium or by the addition of a chelating agent, including EDTA, or related substances, such as citrate. Linnau et al. fail to teach preparations of activated protein C or the effect that metal ions or chelating agents may have on the propensity of activated protein C to form truncated variants or on its stability. Thus, there remains a need in the art to prepare stable, potent formulations of activated protein C. Accordingly, these discoveries allow the preparation of potent in-use aPC formulations to the health care provider that are suitable for administration to a patient in need thereof.

### SUMMARY OF THE INVENTION

The invention provides a pharmaceutical composition comprising aPC and a chelating agent. The pharmaceutical composition contains the chelating agent.

The invention also provides a pharmaceutical composition comprising aPC, a diluent, and a chelating agent. The diluent is either a reconstitution diluent or an intravenous infusion solution wherein the diluent contains the chelating agent.

The invention further provides a pharmaceutical composition in the form of a lyophilized formulation. These pharmaceutical compositions contain aPC, a chelating agent, a bulking agent, a buffer, and/or a salt. Upon reconstitution, the pharmaceutical composition has a pH of about 5.5 to 6.5.

The invention also provides a process for preparing a lyophilized formulation of aPC, which comprises freeze drying a pharmaceutical formulation containing activated protein C, a bulking agent, and a chelating agent.

Also provided is a process of preparing a pharmaceutical solution of aPC, which comprises reconstituting a lyophilized formulation containing activated protein C with a diluent containing a chelating agent.

Further provided is a process of preparing a pharmaceutical solution of aPC, which comprises reconstituting a lyophilized formulation containing activated protein C and a bulking agent with a diluent containing a chelating agent.

Also provided is a method of treating a patient in need thereof which comprises administering to the patient the pharmaceutical composition of the present invention.

Additionally provided is a use of the pharmaceutical composition of the present invention which comprises treating thrombotic disorders.

### DETAILED DESCRIPTION OF THE INVENTION

For purposes of the invention, as disclosed and claimed herein, the following terms are as defined below.

aPC or activated protein C refers to activated protein C whether recombinant or plasma derived. aPC includes and is preferably human activated protein C although aPC may also include other species or derivatives of activated protein C. Examples of protein C derivatives are selected from the group consisting of Gerlitz, et al., U.S. Pat. No. 5,453,373, and Foster, et al., U.S. Pat. No. 5,516,650.

APTT—activated partial thromboplastin time.

r-hPC—recombinant human protein C zymogen.

rhAPC—recombinant human activated protein C.

r-aPC—recombinant activated protein C produced by activating protein C zymogen in vitro or in vivo or by direct secretion of the activated form of protein C from procaryotic cells, eukaryotic cells, or transgenic animals including, for example, secretion from human kidney 293 cells as a zymogen then purified and activated by techniques well known to the skilled artisan. Examples are demonstrated in Yan, U.S. Pat. No. 4,981,952, and Cottingham, WO 97/20043.

Continuous infusion—continuing substantially uninterrupted the introduction of a solution into a blood vessel for a specified period of time.

Bolus injection—the injection of a drug in a defined quantity (called a bolus) at once.

Suitable for administration—a lyophilized formulation or solution that is appropriate to be given as a therapeutic agent.

Zymogen—protein C zymogen, as used herein, refers to secreted, inactive forms, whether one chain or two chains, of protein C.

Pharmaceutically acceptable buffer—a pharmaceutically acceptable buffer is known in the art. Pharmaceutically acceptable buffers include sodium phosphate, sodium citrate, sodium acetate, or TRIS.

Chelating agent—a pharmaceutically acceptable multidentate ligand whose molecules can form several bonds to a single metal ion, creating a ring.

Diluent—a diluent optionally is a reconstitution diluent or an intravenous infusion solution. A reconstitution diluent is a solution used to restore a lyophilized material to the liquid state. An intravenous infusion solution is a solution used as a vehicle for the administration of pharmaceutical compositions or formulations to a patient. A lyophilized formulation is reconstituted prior to its addition to the intravenous infusion solution. Some examples of diluents, as either reconstitution solutions or intravenous infusion solutions, include 0.9% Sodium Chloride, Sodium Chloride with Potassium Chloride, Glucose and Sodium Chloride, 5% Dextrose, Lactated Ringers, 3% Sodium Chloride, Sterile Water for Injection, and Ringers Injection.

EDTA—Ethylenediaminetetraacetic acid that optionally is alone or part of a salt complex. Preferred EDTA forms are selected from the group consisting of dipotassium edetate, disodium edetate, edetate calcium disodium, sodium edetate, trisodium edetate, EDTA free acid, EDTA disodium salt, EDTA disodium salt dihydrate, EDTA disodium-calcium salt, EDTA dipotassium salt, EDTA tripotassium salt EDTA sodium salt, EDTA trisodium salt, EDTA tetrasodium salt hydrate, EDTA tetrasodium salt tetrahydrate, EDTA ferric-sodium salt, EDTA ferric-sodium salt hydrate, and other salts and hydrates. The term edetate is an abbreviation for Ethylenediaminetetraacetic acid.

Activated protein C is an antithrombotic agent with a wider therapeutic index than available anticoagulants, such as heparin and the oral hydroxycoumarin type anticoagulants. Activated protein C is also useful in treating thrombotic disorders. As an antithrombotic agent, aPC has a profound effect on the treatment of a wide variety of acquired disease states involving intravascular coagulation, including thrombotic stroke, deep vein thrombosis, pulmonary embolism, peripheral arterial thrombosis, emboli originating from the heart or peripheral arteries, acute myocardial infarction, disseminated intravascular coagulation, and acute pre or postcapillary occlusions, including transplantations or retina thrombosis. Activated protein C is useful in treating an acquired hypercoagulable state or acquired protein C deficiency associated with sepsis, transplantations, burns, pregnancy, major surgery, trauma, or ARDS. The present formulation provides a method of treating patients by administering a pharmaceutical composition described herein. The present formulation further provides the use of a pharmaceutical composition described herein for treating patients afflicted by one or more of these conditions.

The invention relates to stable formulations and uses of aPC. One stable lyophilized formulation would consist of aPC and a chelating agent. Additionally, a bulking agent is optionally added to the present formulation and selected from the group consisting of mannitol, trehalose, raffinose, and sucrose for use in the lyophilized formulation. Furthermore, the ionic strength is optionally controlled through the salt concentration of the solution. Pharmaceutically acceptable salts typically used to generate ionic strength include but are not limited to potassium chloride (KCl) and sodium chloride (NaCl). The lyophilized product is reconstituted with the appropriate diluent. Preferably, the resulting solution has a pH of about 5.5 to about 6.5. To maintain effective pH control, the aPC solution may contain a pharmaceutically acceptable buffer. Representative buffer systems include Tris-acetate, sodium citrate, and sodium phosphate. A preferable formulation use is a stable lyophilized aPC product that is reconstituted with a diluent containing a chelating agent. Also, a preferable formulation use is a stable lyophilized aPC product that is reconstituted with a diluent then added to an intravenous infusion solution containing a chelating agent or to which a chelating agent will be added.

The molecular interactions in a formulation between aPC, buffer, salt concentration, pH, temperature, and bulking agents, are complex. Yet, the addition of a chelating agent to an aPC formulation with any of the aforementioned components or parameters or to the diluent used with the aPC formulation inhibits less active 1-149 aPC C-terminal light chain variant degradation products. The formulations of the invention provide stable, enzymatically active, aPC with reduced degradation. The invention has reduced or eliminated any increase in the amount of 1-149 aPC C-terminal light chain variant. Preferably, the levels of 1-149 aPC C-terminal light chain variant do not increase by more than 5%. More preferably, 1-149 aPC C-terminal light chain variant levels do not increase by more than 3%. Even more preferably, the levels of 1-149 aPC C-terminal light chain variant do not increase by more than 2%. Most preferably, the levels of 1-149 aPC C-terminal light chain variant do not increase by more than 1%. This stability is obtained through careful control of the processing and formulation conditions and by the addition of a chelating agent.

While the particular source of the aPC is not relevant to the operability of the claimed invention, one illustrative source is depicted in Preparations 1 and 2 below. Preferably the aPC is free of other vitamin-K dependent proteins and factors such as protein S, protein Z, as well as factors II, VU, IX, and X. The formulations described herein are directed to a pharmaceutical product suitable for administration to a patient, either directly, after freeze drying and reconstitution, or, optionally, after dilution in an intravenous infusion solution.

The use of chelating agents for the invention provide a means for sequestering metals that would otherwise promote aPC degradation resulting in the less active 1-149 light chain variant. Representative chelating agents include dipotassium edetate, disodium edetate, edetate calcium disodium, sodium edetate, trisodium edetate, EDTA free acid, EDTA disodium salt, EDTA disodium salt dihydrate, EDTA disodium-calcium salt, EDTA dipotassium salt, EDTA tripotassium salt EDTA sodium salt, EDTA trisodium salt, EDTA tetrasodium salt hydrate, EDTA tetrasodium salt tetrahydrate, EDTA ferric-sodium salt, EDTA ferric-sodium salt hydrate, and other salts and hydrates. The most preferred chelating agent is EDTA disodium salt The skilled artisan will recognize that many other chelating agents are available for use in the formulations of the invention. The optimum concentration of chelating agent is dependent on the amount of metals present in the diluent. Yet, as long as a sufficient concentration of chelating agent is used to sequester all available metals, any excess of chelating agent beyond the necessary amount will have no undue effect on the pharmaceutical composition or formulation. However, the skilled artisan will recognize that the upper range for the amount of chelating agent used is within physiological tolerance. Primarily, the amount of chelating agent is determined based on the aPC concentration. Preferably, the amount of chelating agent is from 1 μM to 10 mM. More preferably, the amount of chelating agent is from 20 μM to 5 mM. The preferred concentration of chelating agent when using 2.5 mg/mL aPC is 1 μM to 10 mM. A more preferred concentration of chelating agent when using 2.5 mg/mL aPC is 20 μM to 5 mM. Even more preferably, the concentration of chelating agent when using 2.5 mg/mL aPC is 50 μM to 1 mM. The most preferred concentration of chelating agent when using 2.5 mg/mL aPC is 500 μM. Alternatively, the preferred concentration of chelating agent when using 5 mg/mL aPC is 1 μM to 10 mM. A more preferred concentration of chelating agent when using 5 mg/mL aPC is 20 μM to 5 mM. Even more preferably, the concentration of chelating agent when using 5 mg/mL aPC is 500 μM to 3 mM. The most preferred concentration of chelating agent when using 5 mg/mL aPC is 1 mM. Using a chelating agent with the diluent containing the formulation stabilizes the aPC for at least 24 hours at room temperature.

Preferred bulking agents in the formulation of aPC are sucrose, trehalose and raffinose. More preferred bulking agents are sucrose and raffinose. The most preferred bulking agent is sucrose. The amount of bulking agent in the formulation is 1 part aPC to 1 to 10 parts bulking agent on a weight-to-weight basis. Moreover, the bulking agent concentration of the formulation is an important formulation variable of the freeze drying process. The optimum concentration of bulking agent is dependent on the amount of aPC and species of bulking agent selected. The preferred concentration of sucrose in the freezing solution is 10 to 40 mg/mL. A more preferred concentration of sucrose is 15 to 30 mg/mL. The most preferred concentration of sucrose in the freezing solution is 15 mg/mL in a formulation of aPC at 2.5 mg/mL. The most preferred concentration of sucrose in the freezing solution is 30 mg/mL in a formulation of aPC at 5.0 mg/mL. The presence of the bulking agent in the formulation of aPC offers increased chemical and physical stability.

Prior to freeze drying and upon reconstitution, it is preferable to maintain the pH in the range of 5.5 to 6.5 to minimize solution state autodegradation. The preferred pH of the formulation is a pH between about pH 5.6 and about pH 6.4. More preferred is a pH between about 5.7 to about 6.3. Even more preferred is a pH between about 5.8 to about 6.2. Still even more preferred is a pH between about 5.9 to about 6.1. The most preferred pH is about pH 6.0.

To maintain effective pH control, the aPC solution may contain a pharmaceutically acceptable buffer. Accordingly, upon freeze-drying, the formulation optionally and preferably comprises a pharmaceutically acceptable buffer. Representative buffer systems include Tris-acetate, sodium citrate, and sodium phosphate. More preferred buffer systems include sodium citrate and sodium phosphate. The most preferred buffer is sodium citrate. The preferred molarity of the buffer system is 10 mM to 50 mM. A more preferred molarity of the buffer system is 10 mM to 20 mM. The most preferred molarity is 40 mM. The skilled artisan will recognize that many other buffer systems are available for use in the formulations of the invention.

Similarly, during freeze drying and upon reconstitution, the ionic strength is controlled to ensure solution state stability. The ionic strength is generally determined by the salt concentration of the solution. Pharmaceutically acceptable salts typically used to generate ionic strength include but are not limited to potassium chloride (KCl) and sodium chloride (NaCl). The preferred salt in the invention is sodium chloride. Preferably, the sodium chloride concentration is greater than 150 mM. More preferably, the sodium chloride concentration in the freezing solution is between 150 mM to 1000 mM. For a formulation containing 2.5 mg/mL aPC, the more preferable sodium chloride concentration in the freezing solution is between 150 mM to 650 mM. Even more preferably the sodium chloride concentration in the freezing solution is between 250 mM to 450 mM. Still even more preferably the sodium chloride concentration in the freezing solution is between 300 mM to 400 mM. The most preferable sodium chloride concentration in the freezing solution is 325 mM for a formulation containing 2.5 mg/mL aPC.

Similarly, for a formulation containing 5.0 mg/mL aPC, the more preferable sodium chloride concentration in the freezing solution is between 150 mM to 1000 mM. Even more preferably the sodium chloride concentration in the freezing solution is between 250 mM to 750 mM. Still even more preferably the sodium chloride concentration in the freezing solution is between 400 mM to 700 mM. The most preferable sodium chloride concentration in the freezing solution is 650 mM for a formulation containing 5.0 mg/mL aPC.

The ratio of aPC:salt:bulking agent (w:w:w) is an important factor in a formulation suitable for the freeze drying process. The ratio varies depending on the concentration of aPC, salt selection and concentration and bulking agent selection and concentration. Particularly, a weight ratio of one part aPC to between about 7 to 8 parts salt to between about 5 to 7 parts bulking agent is preferred. More preferred is a weight ratio of one part aPC to between about 7.5 to about 8 parts salt to between about 5.5 to about 6.5 parts bulking agent. Most preferred is a ratio of about 1 part aPC to about 7.6 parts salt to about 6 parts bulking agent.

The preferred salt is sodium chloride at a concentration of 325 mM (for a formulation containing 2.5 mg/mL aPC) and 650 mM (for a formulation containing 5.0 mg/mL aPC) and at a ratio of about 1.3:1 with sucrose (w:w). This concentration is high enough to cause the salt to crystallize during the freezing process, most likely resulting in an amorphous mixture of aPC, sucrose, and citrate that can be lyophilized. Thus, the ionic strength of NaCl at the preferred concentrations of 325 mM and 650 mM convey stability to the formulation during the freeze-dying process.

The invention also provides a process for preparing a stable lyophilized formulation which comprises lyophilizing a solution comprising aPC and a chelating agent, preferably about 2.5 mg/mL aPC, about 15 mg/mL sucrose, about 19 mg/mL NaCl, about 500 μM EDTA Disodium, and a sodium citrate buffer having a pH greater than 5.5 but less than 6.5. Furthermore, the invention provides a process for preparing a stable lyophilized formulation which comprises lyophilizing a solution comprising about 5 mg/mL aPC, about 30 mg/mL sucrose, about 38 mg/mL NaCl about 1 mM EDTA Disodium, and a citrate buffer having a pH greater than 5.5 but less than 6.5. Such a lyophilization is conducted by routine methods in the art.

Additionally, the invention provides a method of treating disease states involving intravascular coagulation comprising administration of the formulation.

The aPC is preferably administered parenterally to ensure its delivery into the bloodstream in an effective form by injecting the appropriate dose as continuous infusion for about one to about ninety-six hours. The amount of aPC administered is from about 0.01 mg/kg/hr to about 0.05 mg/kg/hr. Alternatively, the aPC will be administered by injecting a portion of the appropriate dose per hour as a bolus injection over a time from about 5 minutes to about 30 minutes, followed by continuous infusion of the appropriate dose for about twenty-three hours to about 47 hours, resulting in the appropriate dose administered over 24 hours to 48 hours.

The following examples will help describe how the invention is practiced and will illustrate the invention. The scope of the invention is not to be construed as merely consisting of the following examples.

### Preparation 1

### Preparation of Human Protein C

Recombinant human protein C (r-hPC) was produced in Human Kidney 293 cells by techniques well known to the skilled artisan such as those set forth in Yan, U.S. Pat. No. 4,981,952, the entire teaching of which is herein incorporated by reference. The gene encoding human protein C is disclosed and claimed in Bang, et al., U.S. Pat. No. 4,775,624, the entire teaching of which is incorporated herein by reference. The plasmid used to express human protein C in 293 cells was plasmid pLPC which is disclosed in Bang, et al., U.S. Pat. No. 4,992,373, the entire teaching of which is incorporated herein by reference. The construction of plasmid pLPC is also described in European Patent Publication No. 0 445 939, and in Grinnell, et al., 1987, Bio/Technology 5:1189-1192, the teachings of which are also incorporated herein by reference. Briefly, the plasmid was transfected into 293 cells, then stable transformants were identified, subcultured and grown in serum-free media. After fermentation, cell-free medium was obtained by microfiltration.

The human protein C was separated from the culture fluid by an adaptation of the techniques of Yan, U.S. Pat. No. 4,981,952. The clarified medium was made 4 mM in EDTA Disodium before it was absorbed to an anion exchange resin (Fast-Flow Q, Pharmacia). After washing with 4 column volumes of 20 mM Tris, 200 mM NaCl, pH 7.4 and 2 column volumes of 20 mM Tris, 150 mM NaCl, pH 7.4, the bound recombinant human protein C zymogen was eluted with 20 mM Tris, 150 mM NaCl, 10 mM CaCl2, pH 7.4. The eluted protein was greater than 95% pure after elution as judged by SDS-polyacrylamide gel electrophoresis.

Further purification of the protein was accomplished by making the protein 3 M in NaCl followed by adsorption to a hydrophobic interaction resin (Toyopearl Phenyl 650 M, TosoHaas) equilibrated in 20 mM Tris, 3 M NaCl, 10 mM CaCl2, pH 7.4. After washing with 2 column volumes of equilibration buffer without CaCl2, the recombinant human protein C was eluted with 20 mM Tris, pH 7.4.

The eluted protein was prepared for activation by removal of residual calcium. The recombinant human protein C was passed over a metal affinity column (Chelex-100, Bio-Rad) to remove calcium and again bound to an anion exchanger (Fast Flow Q, Pharmacia). Both of these columns were arranged in series and equilibrated in 20 mM Tris, 150 mM NaCl, S mM EDTA Disodium, pH 7.4. Following loading of the protein, the Chelex-100 column was washed with one column volume of the same buffer before disconnecting it from the series. The anion exchange column was washed with 3 column volumes of equilibration buffer before eluting the protein with 0.4 M NaCl, 20 mM Tris-acetate, pH 6.5. Protein concentrations of recombinant human protein C and recombinant aPC solutions were measured by UV 280 nm extinction E0.1%=1.81 or 1.85, respectively.

### Preparation 2

### Activation of Recombinant Human Protein C

Bovine thrombin was coupled to Activated CH-Sepharose 4B (Pharmacia) in the presence of 50 mM HEPES, pH 7.5 at 4° C. The coupling reaction was done on resin already packed into a column using approximately 5000 units thrombin/mL resin. The thrombin solution was circulated through the column for approximately 3 hours before adding 2-amino-ethanol (MEA) to a concentration of 0.6 mL/L of circulating solution. The MEA-containing solution was circulated for an additional 10-12 hours to assure complete blockage of the unreacted amines on the resin. Following blocking, the thrombin-coupled resin was washed with 10 column volumes of 1 M NaCl, 20 mM Tris, pH 6.5 to remove all non-specifically bound protein, and was used in activation reactions after equilibrating in activation buffer.

Purified r-hPC was made 5 mM in EDTA Disodium (to chelate any residual calcium) and diluted to a concentration of 2 mg/mL with 20 mM Tris, pH 7.4 or 20 mM Tris-acetate, pH 6.5. This material was passed through a thrombin column equilibrated at 37° C. with 50 mM NaCl and either 20 mM Tris pH 7.4 or 20 mM Tris-acetate pH 6.5. The flow rate was adjusted to allow for approximately 20 min. of contact time between the r-hPC and thrombin resin. The effluent was collected and immediately assayed for amidolytic activity. If the material did not have a specific activity (amidolytic) comparable to an established standard of aPC, it was recycled over the thrombin column to activate the r-hPC to completion. This was followed by 1:1 dilution of the material with 20 mM buffer as above, with a pH of either 7.4 or 6.5 to keep the aPC at lower concentrations while it awaited the next processing step.

Removal of leached thrombin from the aPC material was accomplished by binding the aPC to an anion exchange resin (Fast Flow Q, Pharmacia) equilibrated in activation buffer (either 20 mM Tris, pH 7.4 or 20 mM Tris-acetate, pH 6.5) with 150 mM NaCl. Thrombin does not interact with the anion exchange resin under these conditions, but passes through the column into the sample application effluent. Once the aPC is loaded onto the column, a 2-6 column volume wash with 20 mM equilibration buffer is done before eluting the bound aPC with a step elution using 0.4 M NaCl in either 5 mM Tris-acetate, pH 6.5 or 20 mM Tris, pH 7.4. Higher volume washes of the column facilitated more complete removal of the dodecapeptide. The material eluted from this column was stored either in a frozen solution (−20° C.) or as a lyophilized powder.

The anticoagulant activity of aPC was determined by measuring the prolongation of the clotting time in the activated partial thromboplastin time (APTT) clotting assay. A standard curve was prepared in dilution buffer (1 mg/mL radioimmunoassay grade bovine serum albumin [BSA], 20 mM Tris, pH 7.4, 150 mM NaCl, 0.02% NaN3) ranging in protein C concentration from 125-1000 ng/mL, while samples were prepared at several dilutions in this concentration range. To each sample cuvette, 50 μL of cold horse plasma and 50 μL of reconstituted activated partial thromboplastin time reagent (APTT Reagent, Sigma) were added and incubated at 37° C. for 5 min. After incubation, 50 μL of the appropriate samples or standards were added to each cuvette. Dilution buffer was used in place of sample or standard to determine basal clotting time. The timer of the fibrometer (CoA Screener Hemostasis Analyzer, American Labor) was started immediately after the addition of 50 μL 37° C. 30 mM CaCl2 to each sample or standard. Activated protein C concentration in samples is calculated from the linear regression equation of the standard curve. Clotting times reported here are the average of a minimum of three replicates, including standard curve samples.

### EXAMPLE 1

Intravenous infusion solution stability studies in the vial (˜1-mg/mL) with freshly prepared and filtered 0.9% sodium chloride solution (made at Eli Lilly and Company) containing 5mM, 10 mM, and 20 mM EDTA Disodium were conducted using a rhAPC formulation (made at Eli Lilly and Company; 2 mg/mL aPC, 15.2 mg/mL sucrose, 12 mg/mL NaCl, and a citrate buffer having a pH greater than 5.5 but less than 6.5). The 1-mg/mL I.V. Solutions of rhAPC Formulation prepared in 0.9% sodium chloride solution (made at Eli Lilly and Company), sterile Water for Injection, USP, and in a 150-mL Abbott PVC I.V. bag of 0.9% Sodium Chloride Injection, USP served as controls. The C-Terminal Light Chain Variant (by LC/MS) results from these studies are presented in Tables 1A and 1B.

TABLE 1A
C-Terminal Light Chain Variant Results for ˜1-mg/mL I.V.
Solution of rhAPC Formulation in the Vial with Freshly
Prepared 0.9% Sodium Chloride Solution and sterile Water
for Injection, USP (Control), and in 0.9% Sodium Chloride
Solution Containing 5 mM, 10 mM, and 20 mM EDTA Disodium

| | C-Terminal |
|---|---|
| | Light Chain |
| Sample Description | Variant (%) |
| Initial 1-mg/mL I.V. Solution with | 1-149: ND |
| Freshly Prepared 0.9% Sodium Chloride Solution (Control) | |
| 24-Hour 1-mg/mL I.V. Solution with | 1-149: 59% |
| Freshly Prepared 0.9% Sodium Chloride Solution (Control) | |
| 24-Hour 1-mg/mL I.V. Solution with | 1-149: 19% |
| Sterile Water for Injection (Control) | |
| Initial 1-mg/mL I.V. Solution with | 1-149: ND |
| Freshly Prepared 0.9% Sodium | |
| Chloride Solution Containing | |
| 5 mM EDTA Disodium | |
| 24-Hour 1-mg/mL I.V. Solution with | 1-149: ND |
| Freshly Prepared 0.9% Sodium | |
| Chloride Solution Containing | |
| 5 mM EDTA Disodium | |
| Initial 1-mg/mL I.V. Solution with | 1-149: ND |
| Freshly Prepared 0.9% Sodium | |
| Chloride Solution Containing | |
| 10 mM EDTA Disodium | |
| 24-Hour 1-mg/mL I.V. Solution with | 1-149: ND |
| Freshly Prepared 0.9% Sodium | |
| Chloride Solution Containing | |
| 10 mM EDTA Disodium | |
| Initial 1-mg/mL I.V. Solution with | 1-149: ND |
| Freshly Prepared 0.9% Sodium | |
| Chloride Solution Containing | |
| 20 mM EDTA Disodium | |
| 24-Hour 1-mg/mL I.V. Solution with | 1-149: ND |
| Freshly Prepared 0.9% Sodium | |
| Chloride Solution Containing | |
| 20 mM EDTA Disodium | |

N/A = Not Applicable;
ND = Detection Limit is <2%

TABLE 1B
C-Terminal Light Chain Variant Results for ˜1-mg/mL I.V.
Solution of rhAPC Formulation in Sterile Polypropylene
Tubes and in a 150-mL Abbott PVC I.V. Bag of 0.9% Sodium
Chloride Injection, USP (Control), and in 0.9% Sodium Chloride
Solution Containing 5 mM, 1 mM, 0.2 mM,
0.04 mM EDTA Disodium

| | C-Terminal |
|---|---|
| | Light Chain |
| Sample Description | Variant (%) |
| Initial 1-mg/mL I.V. Solution in a 150-mL | 1-149: 6% |
| PVC I.V. Bag of 0.9% Sodium | |
| Chloride Solution (Control) | |
| 24-Hour 1-mg/mL I.V. Solution in a 150-mL | 1-149: 78% |
| PVC I.V. Bag of 0.9% Sodium | |
| Chloride Solution (Control) | |
| 24-Hour 1-mg/mL I.V. Solution in a 150-mL | 1-149: ND |
| PVC I.V. Bag of 0.9% Sodium Chloride | |
| Solution Containing 5 mM EDTA Disodium | |
| 24-Hour 1-mg/mL I.V. Solution in a 150-mL | 1-149: ND |
| PVC I.V. Bag of 0.9% Sodium Chloride | |
| Solution Containing 1 mM EDTA Disodium | |
| 24-Hour 1-mg/mL I.V. Solution in a 150-mL | 1-149: ND |
| PVC I.V. Bag of 0.9% Sodium Chloride | |
| Solution Containing 0.2 mM EDTA Disodium | |
| 24-Hour 1-mg/mL I.V. Solution in a 150-mL | 1-149: ND |
| PVC I.V. Bag of 0.9% Sodium Chloride | |
| Solution Containing 0.04 mM EDTA Disodium | |

N/A = Not Applicable;
ND = Non-detected.
Detection Limit is <2%

### EXAMPLE 2

Intravenous infusion solution stability studies of a 200-μg/mL rhAPC I.V. solutions in a 150-mL B. Braun/McGaw PAB® I.V. Bag of 0.9% Sodium Chloride Injection, USP (Control) and in 150-mL B. Braun/McGaw PAB® I.V. Bags of 0.9% Sodium Chloride Injection, USP containing 20 μM, 50 μM, and 100 μM, respectively, of EDTA Disodium were conducted using a rhAPC formulation (made at Eli Lilly and Company; 2 mg/mL aPC, 15.2 mg/mL sucrose, 12 mg/mL NaCl, and a citrate buffer having a pH greater than 5.5 but less than 6.5). The rhAPC concentration, potency, and pH results from these studies are presented in Table 2A. The LC/MS results from these studies are presented in Table 2B.

TABLE 2A
rhAPC Concentration, Potency and pH Results for ˜200-μg/mL I.V.
Solutions of rhAPC Formulation in a 150-mL PAB ® I.V. Bag
of 0.9% Sodium Chloride Injection, USP (Control), and in 150-mL
PAB ® I.V. Bags of 0.9% Sodium Chloride Injection, USP
Containing 20 μM, 50 μM, and 100 μM EDTA Disodium

| | rhAPC | | |
|---|---|---|---|
| Sample Description | Conc. | Potency | pH |
| Initial 200-μg/mL I.V. Solution of rhAPC | 210 μg/mL | 456 U/mg | 5.98 |
| Formulation in a 150-mL PAB ® I.V. Bag of 0.9% | | | |
| NaCl Inj. (Control) | | | |
| 8-Hour 200-μg/mL I.V. Solution of rhAPC | 210 μg/mL | 338 U/mg | ND |
| Formulation in a 150-mL PAB ® I.V. Bag of 0.9% | | | |
| NaCl Inj. (Control) | | | |
| 12-Hour 200-μg/mL I.V. Solution of rhAPC | 210 μg/mL | 315 U/mg | ND |
| Formulation in a 150-mL PAB ® I.V. Bag of 0.9% | | | |
| NaCl Inj. (Control) | | | |
| 24-Hour 200-μg/mL I.V. Solution of rhAPC | 210 μg/mL | 249 U/mg | 6.03 |
| Formulation in a 150-mL PAB ® I.V. Bag of 0.9% | | | |
| NaCl Inj. (Control) | | | |
| Initial 200-μg/mL I.V. Solution of rhAPC | 190 μg/mL | 464 U/mg* | 6.00 |
| Formulation in a 150-mL PAB ® I.V. Bag of 0.9% | | | |
| NaCl Inj. Containing 20 μM EDTA Disodium | | | |
| 8-Hour 200-μg/mL I.V. Solution of rhAPC | 190 μg/mL | 450 U/mg* | ND |
| Formulation in a 150-mL PAB ® I.V. Bag of 0.9% | | | |
| NaCl Inj. Containing 20 μM EDTA Disodium | | | |
| 12-Hour 200-μg/mL I.V. Solution of rhAPC | 190 μg/mL | 434 U/mg* | ND |
| Formulation in a 150-mL PAB ® I.V. Bag of 0.9% | | | |
| NaCl Inj. Containing 20 μM EDTA Disodium | | | |
| 24-Hour 200-μg/mL I.V. Solution of rhAPC | 190 μg/mL | 422 U/mg* | 6.01 |
| Formulation t in a 150-mL PAB ® I.V. Bag of 0.9% | | | |
| NaCl Inj. Containing 20 μM EDTA Disodium | | | |
| Initial 200-μg/mL I.V. Solution of rhAPC | 210 μg/mL | 479 U/mg | 5.99 |
| Formulation in a 150-mL PAB ® I.V. Bag of 0.9% | | | |
| NaCl Inj. Containing 50 μM EDTA Disodium | | | |
| 8-Hour 200-μg/mL I.V. Solution of rhAPC | N/A | 461 U/mg | ND |
| Formulation in a 150-mL PAB ® I.V. Bag of 0.9% | | | |
| NaCl Inj. Containing 50 μM EDTA Disodium | | | |
| 12-Hour 200-μg/mL I.V. Solution of rhAPC | N/A | 465 U/mg | ND |
| Formulation in a 150-mL PAB ® I.V. Bag of 0.9% | | | |
| NaCl Inj. Containing 50 μM EDTA Disodium | | | |
| 24-Hour 200-μg/mL I.V. Solution of rhAPC | 220 μg/mL | 428 U/mg | 6.00 |
| Formulation in a 150-mL PAB ® I.V. Bag of 0.9% | | | |
| NaCl Inj. Containing 50 μM EDTA Disodium | | | |
| Initial 200-μg/mL I.V. Solution of rhAPC | 210 μg/mL | 498 U/mg | 5.98 |
| Formulation in a 150-mL PAB ® I.V. Bag of 0.9% | | | |
| NaCl Inj. Containing 100 μM EDTA Disodium | | | |
| 8-Hour 200-μg/mL I.V. Solution of rhAPC | N/A | 475 U/mg | ND |
| Formulation in a 150-mL PAB ® I.V. Bag of 0.9% | | | |
| NaCl Inj. Containing 100 μM EDTA Disodium | | | |
| 12-Hour 200-μg/mL I.V. Solution of rhAPC | N/A | 470 U/mg | ND |
| Formulation in a 150-mL PAB ® I.V. Bag of 0.9% | | | |
| NaCl Inj. Containing 100 μM EDTA Disodium | | | |
| 24-Hour 200-μg/mL I.V. Solution of rhAPC | 220 μg/mL | 451 U/mg | 6.00 |
| Formulation in a 150-mL PAB ® I.V. Bag of 0.9% | | | |
| NaCl Inj. Containing 100 μM EDTA Disodium | | | |

N/A Not Applicable
ND = Not Determined
*The average of three analyses is reported

TABLE 2B
C-Terminal Light Chain Variant Results for ˜200-μg/mL
I.V. Solutions of rhAPC Formulation in a 150-mL PAB ®
I.V. Bag of 0.9% Sodium Chloride Injection, USP (Control), and
in 150-mL PAB ® I.V. Bags of 0.9% Sodium Chloride
Injection, USP Containing 20 μM, 50 μM,
and 100 μM EDTA Disodium

| | C-Terminal |
|---|---|
| | Light Chain |
| Sample Description | Variant (%) |
| Initial 200-μg/mL I.V. Solution of | 1-149: ND |
| rhAPC Formulation in a 150-mL PAB ® I.V. | |
| Bag of 0.9% NaCl Inj. (Control) | |
| 24-Hour 200-μg/mL I.V. Solution of | 1-149: 79% |
| rhAPC Formulation in a 150-mL PAB ® I.V. | |
| Bag of 0.9% NaCl Inj. (Control) | |
| Initial 200-μg/mL I.V. Solution of | 1-149: ND |
| rhAPC Formulation in a 150-mL PAB ® I.V. | |
| Bag of 0.9% NaCl Inj. Containing 20 μM | |
| EDTA Disodium | |
| 24-Hour 200-μg/mL I.V. Solution of | 1-149: ND |
| rhAPC Formulation in a 150-mL PAB ® I.V. | |
| Bag of 0.9% NaCl Inj. Containing 20 μM | |
| EDTA Disodium | |
| Initial 200-μg/mL I.V. Solution of | 1-149: ND |
| rhAPC Formulation in a 150-mL PAB ® I.V. | |
| Bag of 0.9% NaCl Inj. Containing 50 μM | |
| EDTA Disodium | |
| 24-Hour 200-μg/mL I.V. Solution of | 1-149: ND |
| rhAPC Formulation in a 150-mL PAB ® I.V. | |
| Bag of 0.9% NaCl Inj. Containing 50 μM | |
| EDTA Disodium | |
| Initial 200-μg/mL I.V. Solution of | 1-149: ND |
| rhAPC Formulation in a 150-mL PAB ® I.V. | |
| Bag of 0.9% NaCl Inj. Containing 100 μM | |
| EDTA Disodium | |
| 24-Hour 200-μg/mL I.V. Solution of | 1-149: ND |
| rhAPC Formulation in a 150-mL PAB ® I.V. | |
| Bag of 0.9% NaCl Inj. Containing 100 μM EDTA | |
| Disodium | |

N/A = Not Applicable;
ND = Non-detected.
Detection Limit is <2%

### EXAMPLE 3

A stable lyophilized formulation is made by lyophilizing a solution comprising 2.5 mg/mL aPC, 15 mg/mL sucrose, 19 mg/mL NaCl, 500 μM EDTA Disodium, and a sodium citrate buffer having a pH greater than 5.5 but less than 6.5. Also, a stable lyophilized formulation is made by lyophilizing a solution comprising 5 mg/mL aPC, 30 mg/mL sucrose, 38 mg/mL NaCl, 1 mM EDTA Disodium, and a citrate buffer having a pH greater than 5.5 but less than 6.5.

### EXAMPLE 4

A stable lyophilized formulation is made by lyophilizing a solution comprising 2.5 mg/mL aPC, 15 mg/mL sucrose, 19 mg/mL NaCl, and a sodium citrate buffer having a pH greater than 5.5 but less than 6.5. Also, a stable lyophilized formulation is made by lyophilizing a solution comprising 5 mg/mL aPC, 30 mg/mL sucrose, 38 mg/mL NaCl, and a citrate buffer having a pH greater than 5.5 but less than 6.5. Prior to freeze drying and upon reconstitution, the pH is maintained in the range of 5.5 to 6.5. When the lyophilized formulation is reconstituted, the reconstitution diluent contains a sufficient amount of EDTA Disodium to provide 100 μM in the reconstituted solution. Alternatively, the reconstituted formulation is added to a diluent suitable for administration to a patient, such as an intravenous infusion solution, containing a sufficient amount of EDTA Disodium to provide 100 μM in the infusion solution.

1. A pharmaceutical composition comprising activated protein c and a chelating agent.

2. The composition of claim 1 wherein the pharmaceutical composition is a lyophilized formulation

3. The composition of claim 2 further comprising a bulking agent.

4. The composition of claim 3 wherein the bulking agent is selected from the group consisting of mannitol, trehalose, raffinose, and sucrose, and mixtures thereof.

5. The composition of claim 4 further comprising a buffer selected from the group consisting of Tris-acetate, sodium citrate, sodium phosphate, and combinations thereof.

6. The composition of claim 5 further comprising a buffer such that upon reconstitution the formulation has a pH of about 5.5 to about 6.5.

7. The composition of claim 6 further comprising a salt.

8. The composition of claim 7 wherein the salt is selected from the group consisting of potassium chloride and sodium chloride.

9. A pharmaceutical composition comprising activated protein C, a diluent, and a chelating agent.

10. The composition of claim 9 wherein the pharmaceutical composition is a lyophilized formulation.

11. The composition of claim 9 wherein the diluent is a reconstitution diluent.

12. The composition of claim 9 wherein the diluent is an intravenous infusion solution.

13. The composition of claim 9 wherein the chelating agent is present in the diluent.

14. The composition of claim 10 further comprising a bulking agent.

15. The composition of claim 14 wherein the bulking agent is selected from the group, consisting of mannitol, trehalose, raffinose, sucrose, and mixtures thereof.

16. The composition of claim 15 further comprising a buffer selected from the group consisting of Tris-acetate, sodium citrate, sodium phosphate, and combinations thereof.

17. The composition of claim 16 further comprising a buffer such that upon reconstitution the formulation has a pH of about 5.5 to about 6.5.

18. The composition of claim 17 further comprising a salt.

19. The composition of claim 18 wherein the salt is selected from the group consisting of potassium chloride and sodium chloride.

20. A process for preparing a lyophilized formulation of aPC, which comprises freeze drying a pharmaceutical formulation containing activated protein C and a chelating agent.

21. A process for preparing a lyophilized formulation of aPC, which comprises freeze drying a pharmaceutical formulation containing activated protein C, a bulking agent, and a chelating agent.

22. A process of preparing a pharmaceutical solution of aPC, which comprises reconstituting a lyophilized formulation containing activated protein C with a diluent containing a chelating agent.

23. A process of preparing a pharmaceutical solution of aPC, which comprises reconstituting a lyophilized formulation containing activated protein C and a bulking agent with a diluent containing a chelating agent.

24. A method of treating a patient in need thereof which comprises administering to the patient the pharmaceutical composition of any one of claims 1 through 19.

25. (canceled)


SEQUENCE LISTING (3 sequences)

SEQ ID NO: 1 - protein, 410 aa, Homo sapiens
HSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 2 - protein, 409 aa, Homo sapiens
SSLERECIEEICDFEEAKQIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 3 - protein, 419 aa, Homo sapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP